(19) United States
(12) Patent Application Publication
(10) Pub. No.: US 2026/0334995 A1
(43) Pub. Date: Oct. 29, 2026

(54) PLANTAR WART REMOVAL DEVICE

(71) Applicant: DGI Group LLC, Howell, NJ (US)

(72) Inventors: Jeremy Josephson, Lakewood, NJ (US); Zachary Lind, Toms River, NJ (US)

(21) Appl. No.: 19/657,406

(22) Filed: Apr. 24, 2026

Related U.S. Application Data
(60) Provisional application No. 63/794,198, filed on Apr. 24, 2025.

(51) Int. Cl. A61B 18/02

(52) U.S. Cl. CPC A61B 18/0218

(57) ABSTRACT

A plantar wart removal device for initiating removal of a plantar wart includes a canister. The canister holds and dispenses a cryogenic material. The plantar wart removal device includes a housing to house the canister. The plantar wart removal device includes a receptacle configured to cause the canister to selectively dispense the cryogenic material. The plantar wart removal device includes an absorbent tip configured to absorb and store the cryogenic material. The absorbent tip is configured to deposit the cryogenic material onto the plantar wart. The plantar wart removal device includes a tip extender configured to join the housing with the absorbent tip and transmit the cryogenic material therethrough. The tip extender may include a material. The material may be a polymeric material and/or any other suitable material. The absorbent tip is located at a distal portion of the housing.

### CROSS REFERENCE TO RELATED APPLICATIONS

This application is a nonprovisional of U.S. Provisional Application No. 63/794,198 filed on Apr. 24, 2025, which is hereby incorporated by reference herein in its entirety.

### FIELD OF THE DISCLOSURE

Aspects of the disclosure relate to plantar wart removal devices.

### BACKGROUND OF THE DISCLOSURE

Plantar warts, also known as verruca plantaris, are rough, thickened bumps that develop on the bottom of the feet. Plantar warts are typically located on the heels and balls of the feet. Plantar warts can be painful and may cause changes in posture or gait to avoid discomfort. Approximately 1 in every 10 people have plantar warts.

There are chemicals that can be placed on plantar warts to remove the plantar wart. However, because of the location of plantar warts, accurately applying the chemicals is complicated and typically requires a second person to reach the plantar wart for precise removal. Yet a large part of the population typically resists touching other's feet.

Therefore, it would be desirable to create a plantar wart removal device. It would be desirable for the plantar wart removal device to remove the plantar wart by the patient who has the plantar wart. It would be further desirable for the plantar wart removal device to, without the aid of another, precisely apply chemicals to remove the plantar wart.

### SUMMARY OF THE DISCLOSURE

Apparatus, methods and systems for a PLANTAR WART REMOVAL DEVICE is provided.

A plantar wart removal device is provided. The plantar wart removal device may have a longitudinal axis. The plantar wart removal device may enable precise placement and delivery of cryogenic matter. The plantar wart removal device may enable unaided precise placement and delivery of cryogenic material.

The plantar wart removal device may enable unaided placement and delivery of cryogenic material onto plantar warts. As such, the plantar warts may be removed by a patient who has the plantar wart. Removing the plantar wart “unaided” may be understood to refer to a single device user directing all operational controls, such as, but not limited to, controlling the actuator holding the housing, locating the tip of the plantar wart and performing any other suitable operational controls.

The plantar wart removal device may include a canister. The plantar wart removal device may have a longitudinal axis. In certain embodiments, the longitudinal axis may run through the center of the canister. The plantar wart removal device may include a housing. The plantar wart removal device may include an actuator. The plantar wart removal device may include a tip. The plantar wart removal device may include a tip extender.

The plantar wart removal device may include a canister. The canister may include a material. The material may include aluminum. The material may include any other suitable material.

The canister may be a 13-millimeter (“mm”) canister. The canister may be a 20 mm canister. The canister may be any other suitable size canister. The canister may hold one or more wart removal chemicals. The wart removal chemical may be in a liquid form. The wart removal chemical may be in a gas form. The wart removal chemical may be in any other suitable form. The wart removal chemical may turn to gas upon being dispensed from the canister.

The wart removal chemical may be a salicylic acid, cryotherapy agents, tretinoin, glycolic acid, cantharidin, 5-fluorouracil and/or any other suitable chemicals. A cryotherapy agent may include Dimethyl Ether (“DME”) cryogenic solution and/or any other suitable cryogenic agent. It should be noted that although systems and methods are directed towards a wart removal chemical, any other suitable material may be stored within the canister.

In some embodiments, the wart removal chemical may be located in a bag within the canister. As such, the canister may effectively dispense the chemical whether the device and/or canister is upright and/or inverted.

Upon actuating an actuator, the canister may dispense the wart removal chemical. The canister may selectively dispense the wart removal chemical when actuated. The canister may be an inverted canister.

The canister may dispense the wart removal chemical through a gas release mechanism. The gas release mechanism may include a valve. The gas release mechanism may be a stem. The valve may be a pressure relief valve (“PRV”), a metered valve, a continuous feed valve and/or any other suitable valve. The metered valve may be a metered dose valve. The canister may release the wart removal chemical using the metered dose valve. The canister may release the wart removal chemical using the metered dose valve.

In some embodiments, the plantar wart removal device may include a mechanism for measuring release of gas from a continuous valve. The mechanism may deliver more gas than a metered dose valve, while simultaneously mitigating gas loss. The mechanism may thereby ensure, during wart removal treatment, an optimal lesion treatment temperature, because an optimal lesion treatment temperature is maintained during treatment. An optimal lesion treatment temperature may be, for example, a temperature between −57 °C and −196 °C. Additionally, the mechanism may minimize the number of treatments needed.

The mechanism may include a triangular shelf. The triangular shelf may include a slightly rounded top. The shelf may enable and/or make it easier for the gas to slide off the gas release stem during activation of the canister. The gas may only be released when the shelf facilitates the gas slide off the gas released mechanism and be released. The amount of time it takes for the gas to slide down the shelf may affect how long the stem remains open. As such, the gas is released according to the amount of time required to create the optimal treatment for the lesion.

The plantar wart removal device may include a housing. The housing may include a material. The material may be a plastic. The plastic may be a medical grade molded plastic. Medical grade molded plastic may include polyethylene, polypropylene, polyvinyl, polyvinyl chloride, polystyrene, polycarbonate, acrylonitrile butadiene styrene, polyetheretherketone, acrylic, polyethylene terephthalate glycol, any combination of the foregoing and/or any other suitable medical grade molded plastics. The housing may include any other suitable material.

The housing may house the canister. The housing may be hollow inside. The housing may house the canister. The canister may be placed in the hollow portion of the housing.

In some embodiments, the housing may include a collar. The collar may be located towards a distal portion of the housing. Preferably, the collar may be located on the housing immediately less distal than, or in certain embodiments, below, i.e., more distal than, a tip extender. The collar may include a chrome finish. The chrome finish may be a chrome plated parabolic mirror surface and/or any other suitable chrome finish. The chrome finish may reflect. As such, when the tip is being applied to or is located near a plantar wart, the collar may ensure sighting of the wart through a reflection on the collar. The collar may ensure precise placement of the device on the plantar wart.

In some embodiments, the housing may include a gripping portion. The gripping portion may be located at a distal portion of the housing. The gripping portion may be a horizontal gripping portion. The gripping portion may provide a comfortable grip for a user. The gripping portion may be suitable to be held by a single hand. The gripping portion may be located either above or below an adductor pollicis. An adductor pollicis, for purposes of the application, is a muscle that adducts the thumb, bringing the thumb toward the index finger.

The plantar wart removal device may include an actuator. The plantar wart removal device may include one or more actuators. In some embodiments, the one or more actuators may be mounted on the housing. It should be noted that the one or more actuators may preferably be a button. When the button is selected, pressed, depressed and/or otherwise activated, the canister may dispense the wart removal chemical.

The one or more actuators may be mounted on a proximal portion of the housing. Such actuators may be suitable for thumb actuation. The one or more actuators may be mounted on one or more sides of the housing. Such actuators may be suitable for full hand actuation. As such, a full hand may be used for actuating the one or more actuators.

In some embodiments, two actuators may be used. The two actuators may be located towards a distal portion of the housing. The two actuators may be opposed buttons. The two actuators may be pinched together at the same time. Such actuators may be suitable for actuation, for example, by a thumb and an index finger of a single hand. When the two actuators are pinched together, the canister may dispense the wart removal chemical. In other embodiments, the housing may include a portion for placement of the one or more actuators.

In some embodiments, the canister may be an upright canister. In such embodiments, the canister may include an internal sipper tube. In such embodiments, one or more actuators may preferably be located at one or more sides of the housing. In such embodiments, the tip may be located at a proximal portion of the housing.

A location of the one or more actuators may determine how the plantar wart removal device is held. When the actuators are located towards a distal portion of the device, a user may hold the device with a pincer grasp. A pincer grasp, for purposes of the application, may be understood to refer to bringing the thumb and the index finger into precise opposition. A pincer grasp may enable optimal control and accurate positioning.

When the actuator is located at a proximal portion of the device, a user may hold the device with a pipette grasp. A pipette grasp may allow a palm to firmly grip the device. However, a pipette grasp may have less control and positioning accuracy than a pincer grasp.

In some embodiments, a plier grasp may be used. The actuators may include wings that may be held together. Preferably, such a grasp may include an upright canister. As such, grasping the device may be comfortable. Furthermore, grasping the device may enable stable symmetrical tip positioning.

In other embodiments, a palm grip handle may be used. The palm grip handle may preferably include two actuators. In such embodiments, the device may be held upright when removing the plantar wart. As such, the canister may be an upright canister.

In still other embodiments, the actuator may be distinct from the plantar wart removal device. In such embodiments, the actuator may be referred to as an activation base receptacle. The activation base receptacle may receive the plantar wart removal device. The plantar wart removal device may be pressed down into the activation base receptacle. When the device is pressed down into the activation base receptacle, the device may dispense the wart removal chemical.

The plantar wart removal device may include a tip extender. The tip extender is configured to enable delivery of medication in a way that is more convenient and precise than just using the housing and canister alone.

The tip extender may join the housing with the tip. The tip extender may be formed from the same material as the housing. As such, the tip extender may be formed from a medical grade molded plastic. The tip extender may be formed from a thermoplastic elastomer. The tip extender may be formed from any other suitable material. The tip extender may be formed from a different material than the housing. The tip extender may be located at a distal portion of the housing.

The tip extender may transmit the wart removal chemical therethrough either as a gas or as a liquid. The tip extender may be connected to the to the canister at a proximal portion. The tip extender may be connected to the tip at a distal portion.

The tip extender may have a tip extender longitudinal axis. The tip extender longitudinal axis may be between a 30- and 90-degreee angle with the longitudinal axis, the housing, the canister and/or the device. As such, the tip extender may extend from the housing at between a 30- and 90-degree angle.

The tip extender may be bendable. When the tip extender may be formed from thermoplastic elastomer, the tip extender may include a corrugated configuration. As such, the bendability of the tip extender may enable precise positioning of the device with respect to the plantar wart. The tip extender may be bendable for precisely targeting the plantar wart.

In embodiments where the tip extender is bendable, the tip extender may include a circumferential stabilizing support pad. The circumferential stabilizing support pad may be distinct from the support pad. The circumferential stabilizing support pad may be formed from a flexible elastomer. The circumferential stabilizing support pad may be formed from any other suitable material. The circumferential stabilizing support pad may be transparent. The circumferential stabilizing support pad may be used to maintain positioning. Specifically, the circumferential stabilizing support pad may maintain a perpendicular position of the plantar wart removal device. It should be noted that the circumferential stabilizing support pad may be removed from the device.

The tip extender may include a conduit for transfer of the wart removal chemical from the canister. The conduit may extend from a distal end of the canister to the tip. The wart removal chemical may pass through the conduit to the tip.

The plantar wart removal device may include a tip. In some embodiments, the tip may be formed from an absorbent material. The tip may contact the plantar wart. The tip may be formed from a thermally conductive material, an absorbent material and/or any other suitable material. The conductive material may be sintered and/or any other suitable conductive material. Preferably, the tip may be formed from an absorbent material. The absorbent material may be a foam, an open cell foam, a reticulated foam and/or any other suitable absorbent material. The reticulated foam may be an 80 pores per inch (“PPI”) and/or any other suitable size.

The tip may include a diameter. The diameter of the absorbent tip may be different than the diameter of a metal tip. The diameter of the absorbent tip may be 6 mm. The diameter of the absorbent tip may be between 5 and 7 millimeters. The diameter of the absorbent tip may be any other suitable size. The absorbent tip may include a depth. The depth may be between 6 and 10 millimeters. The depth may be any other suitable depth.

The metal tip may include a diameter. The diameter of the metal tip may be 3 mm, about 3 mm, more than 3 mm or less than 3 mm. The diameter of the metal tip may be any other suitable diameter.

The tip may receive the wart removal chemical from the canister, via the tip extender. The tip may absorb the wart removal chemical. The tip may become saturated with the wart removal chemical. The tip may contact the plantar wart. The tip may contact the plantar wart for a duration of between 15 and 25 seconds. The tip may contact the plantar wart for any other suitable amount of time. The tip may be compressed when pressed down onto the plantar wart. Alternatively, the tip may be mounted onto the canister and gas released from the canister may be channeled through the tip extender to wart.

The tip may become saturated with the wart removal chemical. When the tip contacts the plantar wart, the tip may deposit the wart removal chemical onto the plantar wart. When the tip deposits the wart removal chemical onto the plantar wart, the wart removal chemical may freeze the plantar wart. Preferably, the tip may freeze the plantar wart for a duration of between 15 and 25 seconds. The tip may freeze the plantar wart for any other suitable amount of time.

Preferably, the tip may deposit the wart removal chemical onto the plantar wart for a 15-25 second duration, or more, a 20-30 second duration, or more and/or any other suitable duration of time. Thereafter, the wart may be allowed to thaw for a suitable amount of time, such as 20-40 seconds, or more. Then, the plantar wart may be refrozen for an additional 15-25 seconds, or more. The duration of contact between the tip and the plantar wart may be between according to the size of the plantar wart. The tip may deposit the wart removal chemical onto the plantar wart for any other suitable amount of time. In some embodiments, the amount of time the tip contacts the plantar wart may be determined based on a size of the plantar wart.

In embodiments where the actuator is distinct from the plantar wart removal device, the tip may be an absorbent tip. The plantar wart removal device may be placed inside the activation base receptacle. When the plantar wart removal device is pressed into the activation base receptacle, a gas release mechanism within the canister may be activated. The gas release mechanism may release the wart removal chemical from the canister.

In certain embodiments, the wart removal chemical may be released from the canister into the tip. The activation base receptacle may ensure that the tip is fully saturated. The fully saturated tip may be used for venting the released gas. The fully saturated tip may enable overspray gas released from the tip onto the plantar wart to be transmitted back onto the tip. As such, the treatment tip may include a decreased cooling temperature and longer cooling retention than commonly used for removal of common warts. Such a device may be suitable for removal of plantar warts because plantar warts require a longer and colder treatment cycle than common warts.

The metal tip may receive the wart removal chemical from the canister. The canister may include a fluid conduit. The conduit may be a thin tube. The conduit may transmit the wart removal chemical from the canister, through the tip extender, to the tip.

In some embodiments, a skin patch may be placed around the plantar wart. The skin patch may be a stick-on patch. The skin patch may be a removable patch. The skin patch may be formed from a mini-cell closed cell foam. A mini-cell closed cell foam may be referred to as a crosslink polyethylene foam. The mini-cell closed cell foam may have a smooth surface. The mini-cell closed cell foam may provide flexibility and resilience. The skin patch may be formed from any other suitable material. The skin patch may include an adhesive backing. The adhesive backing may be a high tack polyurethane adhesive. The skin patch may be any other suitable skin patch.

The skin patch may include an aperture. The aperture may be situated on the plantar wart. The aperture may enable the plantar wart removal device to fit into the skin patch. The aperture may enable the skin patch to maintain the position of the plantar wart removal device on the plantar wart.

The skin patch may protect skin adjacent to the plantar wart from the cryogenic material. As such, the skin adjacent to the plantar wart may be prevented from being frozen or otherwise by the cryogenic material. The skin patch may enable the plantar wart removal device to be repositioned for additional treatment-e.g., the second freezing cycle mention above.

The skin patch may include a tab. The tab may be a pull tab. The tab may not include the adhesive backing. The tab may be used to remove the skin patch.

### BRIEF DESCRIPTION OF THE DRAWINGS

The objects and advantages of the disclosure will be apparent upon consideration of the following detailed description, taken in conjunction with the accompanying drawings, in which like reference characters refer to like parts throughout, and in which:

FIGS. 1A-1B show illustrative diagrams in accordance with the principles of the disclosure;

FIG. 2A-2B show illustrative diagrams in accordance with the principles of the disclosure;

FIG. 3A-3C show illustrative diagrams in accordance with the principles of the disclosure;

FIG. 4A-4B show illustrative diagrams in accordance with the principles of the disclosure;

FIG. 5A-5B show illustrative diagrams in accordance with the principles of the disclosure;

FIG. 6A-6B show illustrative diagrams in accordance with the principles of the disclosure;

FIG. 7A-7B show illustrative diagrams in accordance with the principles of the disclosure;

FIG. 8 shows an illustrative diagram in accordance with the principles of the disclosure;

FIG. 9 shows an illustrative diagram in accordance with the principles of the disclosure;

FIG. 10 shows an illustrative diagram in accordance with the principles of the disclosure;

FIG. 11 shows an illustrative diagram in accordance with the principles of the disclosure;

FIG. 12 shows an illustrative diagram in accordance with the principles of the disclosure;

FIG. 13A-13D show illustrative diagrams in accordance with the principles of the disclosure;

FIG. 14 shows an illustrative diagram in accordance with the principles of the disclosure; and

FIG. 15 shows an illustrative diagram in accordance with the principles of the disclosure.

### DETAILED DESCRIPTION OF THE DISCLOSURE

Apparatus, methods and systems for a PLANTAR WART REMOVAL DEVICE are provided.

A plantar wart removal method is provided. The plantar wart removal method may use a plantar wart removal device.

The plantar wart removal method may include holding and selectively dispensing a cryogenic material. Although methods and systems are directed to cryogenic material, any other suitable wart removal chemical may be used. The cryogenic material may be stored in a canister within the plantar wart removal device. The plantar wart removal device may have a longitudinal axis. The longitudinal axis may run through the center of the device. In certain embodiments, the longitudinal axis may run through the center of the canister.

The canister may include a material. The material may be aluminum, metal and/or any other suitable material. The canister may store the cryogenic material in a pressurized liquid form. The canister may be sealed. When a valve, located within the canister, is opened, the canister may release the cryogenic material. The cryogenic material may turn to a gas upon being dispensed and/or released from the canister.

The valve may be a pressure relief valve (“PRV”), a metered valve, a continuous release valve and/or any other suitable valve. The metered valve may be a metered dose valve. The valve may be opened. When the valve is opened, the canister may release the cryogenic material.

The canister may be located within a housing. The housing may include a material. The material may be a medical grade molded plastic and/or any other suitable plastic. The material may be any other suitable material.

The cryogenic material may be stored in the canister pre-application. Pre-application, for purposes of the embodiment, may be understood to refer to prior to the use of the plantar wart removal device.

The plantar wart removal method may include causing the canister to selectively dispense the cryogenic material when activated. The plantar wart removal device may include an actuator. The actuator may be located on the housing. The actuator may be distinct from the plantar wart removal device. The actuator may be a button, a receptacle and/or any other suitable actuator.

When the actuator is pressed, depressed and/or actuated in any other way, the actuator may cause the canister to dispense the cryogenic material. The canister may dispense the cryogenic material through the valve.

The plantar wart removal method may include absorbing the cryogenic material in an absorbent tip. Although directed to an absorbent tip, the tip may include and/or be formed from any other suitable material. The absorbent tip may form a part of the plantar wart removal device. The cryogenic material may be transmitted from the canister to the absorbent tip via a tip extender.

It should be noted that the method may include fully saturating the absorbent tip. The actuator and/or receptacle may ensure that the tip is fully saturated. Pressing and/or depressing the actuator for a predetermined amount of time may ensure that the tip is fully saturated. The valve may ensure that the tip is fully saturated by releasing a predetermined amount of cryogenic matter onto the tip. Any other suitable method may be used to ensure that the tip is fully saturated.

The plantar wart removal method may include storing the cryogenic material in the absorbent tip. After the tip absorbs the cryogenic material, the tip may store the cryogenic material. The tip may store the plantar wart removal device until the tip is placed on the plantar wart.

The plantar wart removal method may include applying the absorbent tip to the plantar wart to freeze the plantar wart. The absorbent tip may be applied to the plantar wart for between 15 and 25 seconds, or more. The absorbent tip may be applied to the plantar wart for any other suitable duration of time. When the absorbent tip is applied to, or near, the plantar wart, the cryogenic material, located within the absorbent tip may freeze the plantar wart.

It should be noted that the plantar wart removal device may be configured to automatically maintain the absorbent tip on the plantar wart for a predetermined amount of time. Preferably, the predetermined amount of time may be between 15 and 25 seconds. The predetermined amount of time may be any other suitable amount of time. It should be noted that the predetermined amount of time may be determined based on a size of the plantar wart. The plantar wart removal device may be electronically configured to maintain the plantar wart removal device on the plantar wart for the predetermined amount of time. The plantar wart removal device may be otherwise configured to maintain the plantar wart removal device on the plantar wart for the predetermined amount of time.

The plantar wart removal method may include allowing the plantar wart to thaw. The plantar wart removal device may be configured to thaw the plantar wart for a duration of time. The duration of time may be a predetermined amount of time. Preferably, the predetermined amount of time may be between 20 and 40 seconds, or more. The predetermined amount of time may be any other suitable amount of time. It should be noted that the predetermined amount of time may be determined based on a size of the plantar wart.

The plantar wart removal device may be configured to automatically allow the plantar wart to thaw for the predetermined amount of time. The plantar wart removal device may retract the absorbent tip into the plantar wart removal device. The cryogenic matter may be retracted into the plantar wart removal device. The plantar wart removal device may be configured to automatically thaw the plantar wart using any other suitable method. The plantar wart removal device may be electronically configured to allow the plantar wart to thaw for the predetermined amount of time.

The plantar wart removal method may include reapplying the absorbent tip to refreeze the plantar wart. The plantar wart removal method may reapply the absorbent tip to refreeze the plantar wart for a duration of between 15 and 25 seconds or more. The plantar wart removal method may reapply the absorbent tip to refreeze the plantar wart for any other suitable duration of time. The plantar wart removal device may be configured to automatically reapply the absorbent tip to refreeze the plantar wart. The plantar wart removal device may be electronically configured to automatically reapply the absorbent tip to refreeze the plantar wart.

Illustrative embodiments of methods, systems, and apparatus in accordance with the principles of the invention will now be described with reference to the accompanying drawings, which form a part hereof. It is to be understood that other embodiments may be used, and structural, functional, and procedural modifications may be made without departing from the scope and spirit of the present invention.

The drawings show illustrative features of methods, systems, and apparatus in accordance with the principles of the invention. The features are illustrated in the context of selected embodiments. It will be understood that features shown in connection with one of the embodiments may be practiced in accordance with the principles of the invention along with features shown in connection with another of the embodiments.

The methods, apparatus, computer program products, and systems described herein are illustrative and may involve some or all the steps of the illustrative methods and/or some or all of the features of the illustrative system or apparatus. The steps of the methods may be performed in an order other than the order shown or described herein. Some embodiments may omit steps shown or described in connection with the illustrative methods. Some embodiments may include steps that are not shown or described in connection with the illustrative methods but rather are shown or described in a different portion of the specification.

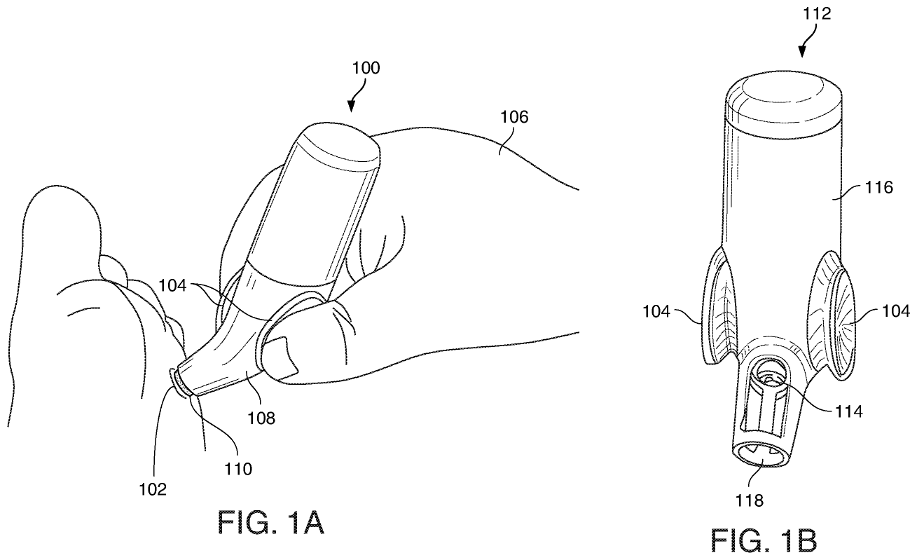

FIG. 1A shows an illustrative diagram. The illustrative diagram includes an image of a user 106 using a plantar wart removal device 100 to initiate removal of a plantar wart 102 from user 106's foot. It should be noted that a plantar wart removal device 100 is preferably configured to be used single-handedly and unaided by a user, but could be used by one person on another person's plantar wart. It should be further noted that the user may remove the plantar wart from a user's own foot.

Plantar wart removal device 100 may include a housing. The housing may be shown as housing 116 in FIG. 1B and described in the portion of the specification corresponding thereto.

Plantar wart removal device 100 may include a canister. The canister may include a wart removal chemical. The wart removal chemical may be in a liquid form. The wart removal chemical may be in a gas form. The wart removal chemical may be cryogenic material and/or any other suitable Wart removal chemical. The cryogenic material may be a Dimethyl Ether (“DME”) cryogenic solution and/or any other suitable cryogenic material.

Plantar wart removal device 100 may include one or more actuators 104. Actuators 104 may be buttons. Actuators 104 may be opposed buttons for pinch actuation. When actuators 104 are pinched, or otherwise actuated, the canister may dispense the wart removal chemical. The canister may selectively dispense the wart removal chemical.

Plantar wart removal device 100 may include a tip 110. Tip 110 may be formed from a conductive material, an absorbent material and/or any other suitable material. Tip 110 may receive the wart removal chemical from the canister. Tip 110 may absorb the wart removal chemical. Tip 110 may store the wart removal chemical. Tip 110 may be applied to and/or located near plantar wart 102. Tip 110 may deposit the wart removal chemical onto plantar wart 102.

Tip 110 may freeze plantar wart 102 when applied to plantar wart 102. Tip 110 may be applied to plantar wart 102 for a duration of 15-25 seconds, or more. Tip 110 may be applied to plantar wart 102 for any other suitable duration of time.

Plantar wart removal device 100 may be removed from plantar wart 102 after 15-25 seconds, or more. As such, plantar wart 102 may be allowed to thaw. Plantar wart 102 may be allowed to thaw for 20-40 seconds, or more. Plantar wart 102 may be allowed to thaw for any other suitable duration of time.

Plantar wart removal device 100 may be reapplied to plantar wart 102. Plantar wart removal device 100 may refreeze plantar wart 102 for a duration of 15-25 seconds, or more. As such, plantar wart 102 may die and may fall off shortly thereafter.

FIG. 1B shows an illustrative diagram. FIG. 1B shows plantar wart removal device 112. Plantar wart removal device 112 may be similar to plantar wart removal device 100.

Plantar wart removal device 112 may include housing 116. The housing may include a material. The material may include a medical grade molded plastic. The material may include any other suitable material.

However, in place of tip 110 and tip extender 108, plantar wart removal device 112 may include tip nozzle 114 and tip enclosure 118. Tip nozzle 114 may include a material. The material may be plastic, metal and/or any other suitable material. Tip enclosure 118 may include a material. The material may include translucent plastic, plastic and/or any other suitable material. Tip nozzle 114 may be a spray nozzle.

Plantar wart removal device 112 may include a liquid wart removal chemical within the canister. Plantar wart removal device 112 may be placed on top of a plantar wart using tip enclosure 118. Actuators 104 may actuate plantar wart removal device 112 to spray the wart removal chemical out of tip nozzle 114, through tip enclosure 118 and onto a plantar wart. Tip nozzle 114 may focus the output, preferably the wart removal chemical, from tip enclosure 118. Although the wart removal chemical is stored within the canister as a liquid, the wart removal chemical may spray out as a gas.

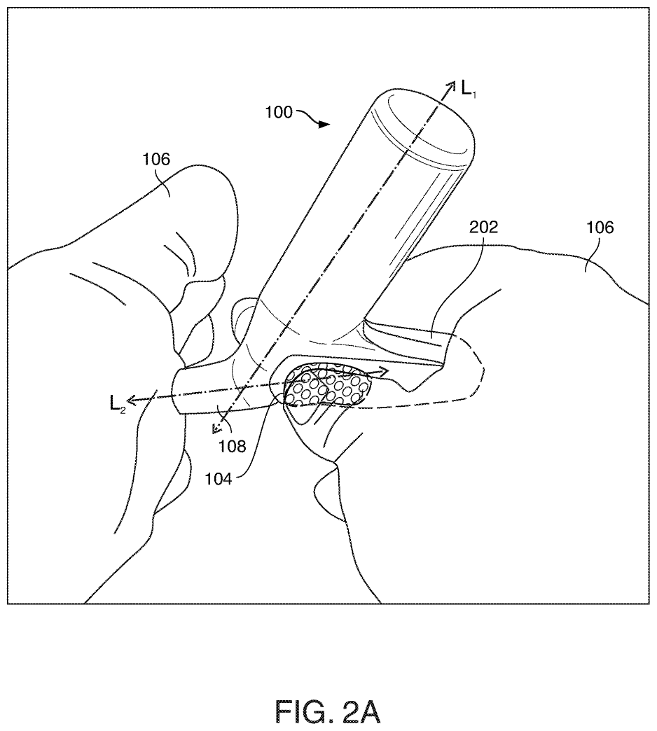

FIG. 2B shows an illustrative diagram. Illustrative diagram 2B shows plantar wart removal device 100 initiating a removal of a plantar wart located on the balls of user 106's feet. Plantar wart removal device 100 may include longitudinal axis L₁. Longitudinal axis L₁ may run through the center of the housing of plantar wart removal device 100. In certain embodiments, longitudinal axis L₁ may run through the center of the canister.

Plantar wart removal device 100 may include tip extender 108. Tip extender 108 may include tip extender longitudinal axis L₂. Tip extender longitudinal axis L₂ may form between a 30- and 90-angle with longitudinal axis L₁. As such, tip extender 108 may be between a 30- and 90-degree angle with housing 116. Because tip extender 108 is located between a 30- and 90-degree angle with housing 116, user 106 may precisely position plantar wart removal device 100 on the plantar wart.

It should be noted that plantar wart removal device shown in FIG. 2B may include horizontal gripping portion 202. Horizontal gripping portion 202 may enable comfortable grip while holding the device. Horizontal gripping portion 202 may include actuators 104. Horizontal gripping portion 202 may be held either above or below an abductor pollicis longus is an extrinsic muscle of the thumb, located in the deep posterior compartment of the forearm.

A thumb and an index finger of the same hand may pinch actuators 104 together. When actuators 104 are pinched together, plantar wart removal device 100 may dispense the wart removal material.

FIG. 2B shows an illustrative diagram. FIG. 2B is similar to 2A. However, FIG. 2B shows a plantar wart removal device 100 initiating a removal of a plantar wart on the heels of user 106's feet.

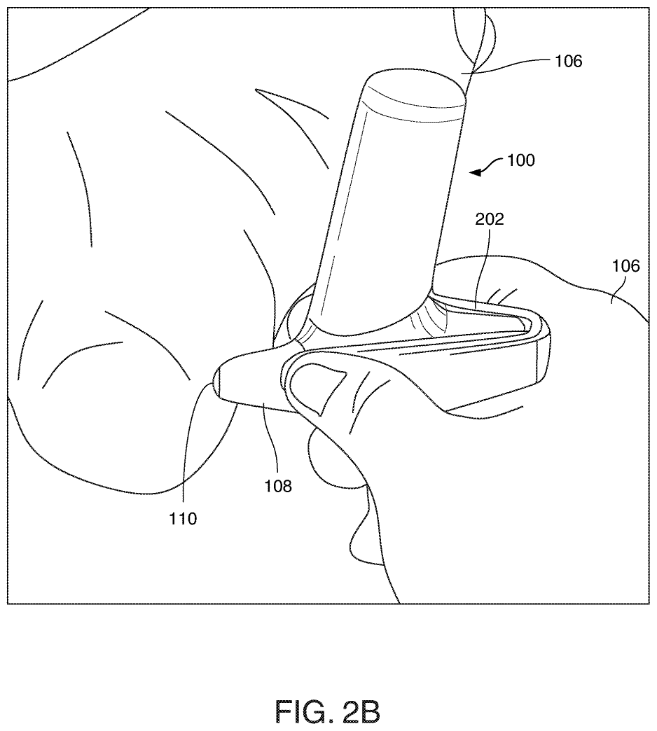

FIG. 3A shows an illustrative diagram. FIG. 3A shows an alternative plantar wart removal device. The alternative plantar wart removal device shown in FIG. 3A includes housing 306, handle support 302 and stability support 304. Such a plantar wart removal device may be preferred. Such a plantar wart removal device may provide additional support to a user.

FIG. 3B shows an illustrative diagram. FIG. 3B shows an alternative plantar wart removal device. The alternative plantar wart removal device shown in FIG. 3B includes housing 306, handle support 302 and stability support 304. Such a plantar wart removal device may be preferred. Such a plantar wart removal device may provide additional support to a user.

FIG. 3C shows an illustrative diagram. FIG. 3C shows an alternative plantar wart removal device. The alternative plantar wart removal device shown in FIG. 3A includes housing 306, handle support 302 and stability support 304. Such a plantar wart removal device may be preferred. Such a plantar wart removal device may provide additional support to a user.

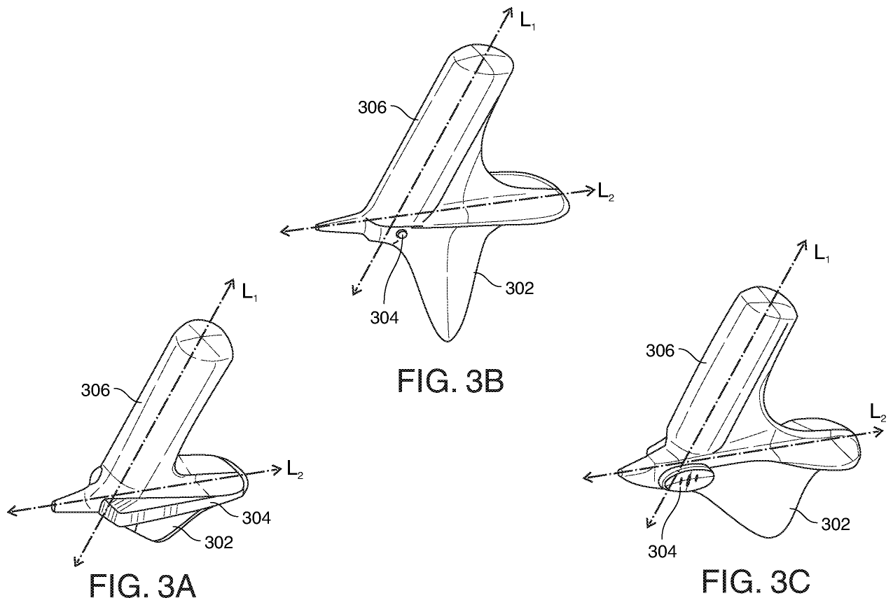

FIG. 4A shows an illustrative diagram. The illustrative diagram shows an initiation of removal of a plantar wart using plantar wart removal device 400. Plantar wart removal device 400 may relate to plantar wart removal device 100, shown at least in FIG. 1A and described in the portion of the specification corresponding thereto.

Plantar wart removal device 400 may include canister 402. Plantar wart removal device 400 may be a thumb actuated device. User 106 may press canister 402 down with a thumb and/or using any other suitable method. When canister 402 is pressed, depressed or otherwise actuated, canister 402 may release a wart removal chemical, stored therein. The wart removal chemical may be transmitted to a tip. The tip may absorb and store the wart removal chemical. The tip may deposit the wart removal chemical onto the plantar wart.

FIG. 4B shows a perspective view of plantar wart removal device 400.

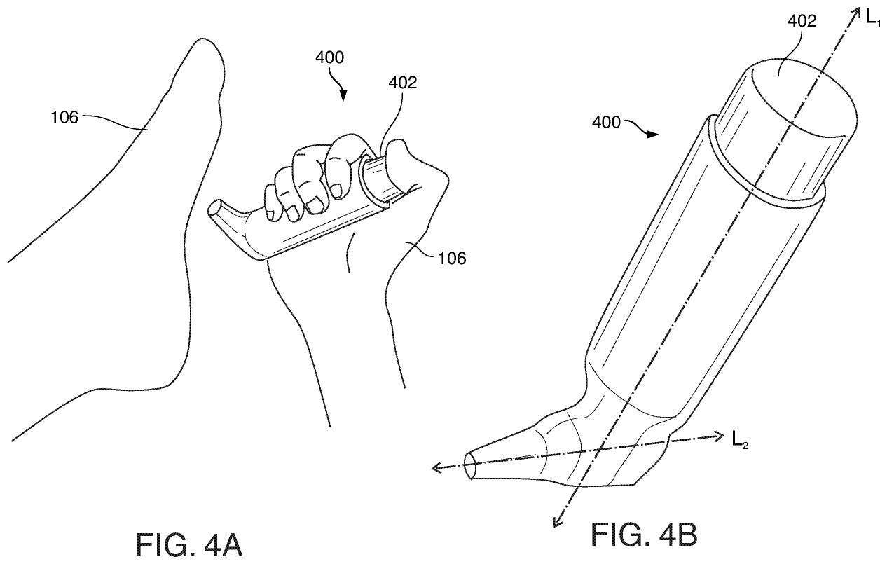

FIG. 5A shows an illustrative diagram. FIG. 5A shows plantar wart removal device 502. Plantar wart removal device 502 is a full hand grip actuated plantar wart removal device. As such, a full hand may be used to actuate the plantar wart removal device to dispense the wart removal chemical.

FIG. 5B shows another illustrative diagram. FIG. 5B shows an alternative full hand grip actuated plantar wart removal device. FIG. 5B shows plantar wart removal device 504. Plantar wart removal device 504 may include actuators 506. Actuators 506 may be full hand grip actuators.

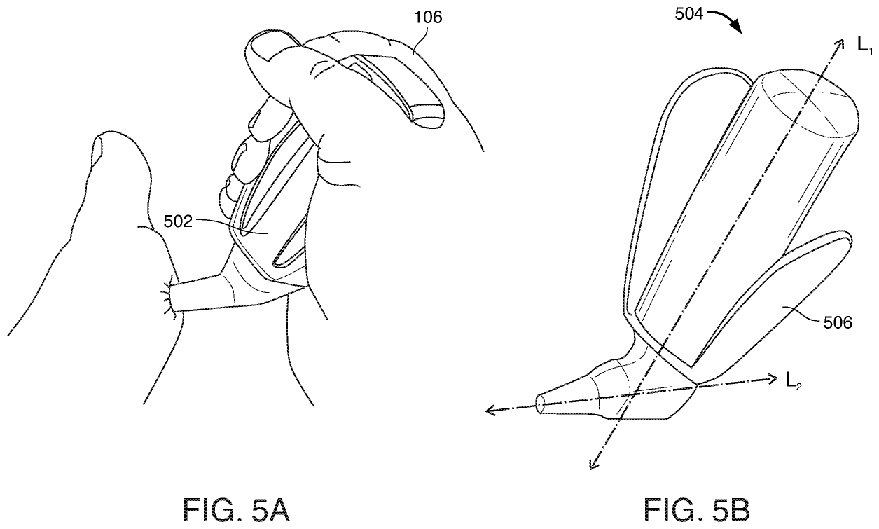

FIG. 6A shows an illustrative diagram. FIG. 6A shows a plantar wart removal device 600. Plantar wart removal device 600 includes tip 602, tip extender 604, actuator 606 and housing 608. Plantar wart removal device 600 may be similar to plantar wart removal device 100. However, plantar wart removal device 600 may be designed to be used when held upright. Plantar wart removal device 600 may be preferred by left-handed users. Plantar wart removal device 600 may be a thumb actuated device. Actuator 606 may be pressed or depressed with a thumb and/or with any other suitable method.

FIG. 6B shows an illustrative diagram. FIG. 6B shows plantar wart removal device 610. Plantar wart removal device 610 may include tip 612, tip extender 614, actuators 616 and housing 618. Plantar wart removal device 610 may be a palm actuated device. Actuators 616 may be pressed or depressed with a palm and/or with any other suitable method. Plantar wart removal device 610 may be designed to be used when held upright. Plantar wart removal device 610 may be preferred by left-handed users.

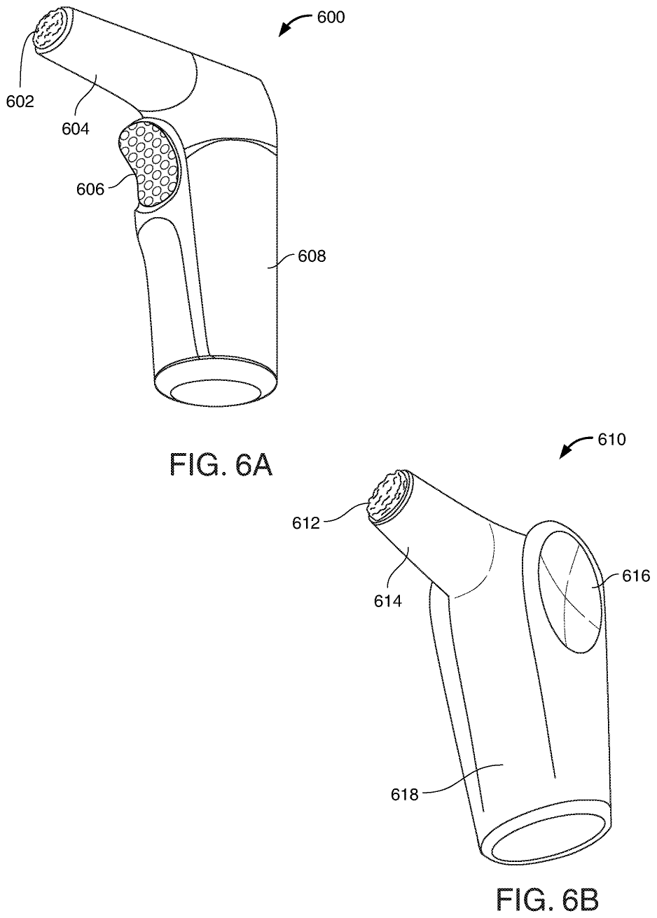

FIG. 7A shows illustrative diagram 700. Illustrative diagram 700 includes a plantar wart removal device 702. Plantar wart removal device 702 is suitable for a pliers' type grip. However, squeezing a pliers' type grip with an inverted hand orientation can be awkward, fatiguing and cause device tip instability.

FIG. 7B shows illustrative diagram 706. Illustrative diagram 706 includes a plantar wart removal device 708. Plantar wart removal device 708 shows a conventionally held pliers' grip. A thumb and an opposed index finger are pinched together to actuate plantar wart removal device 708. Such a pliers' grip may be more comfortable and enable a stable symmetrical tip position.

Plantar wart removal device 708 may include a canister. The canister may utilize a bag-in-bottle technology. The bag-in-bottle may enable effective spray of a wart removal chemical in both an upright position as well as an inverted position.

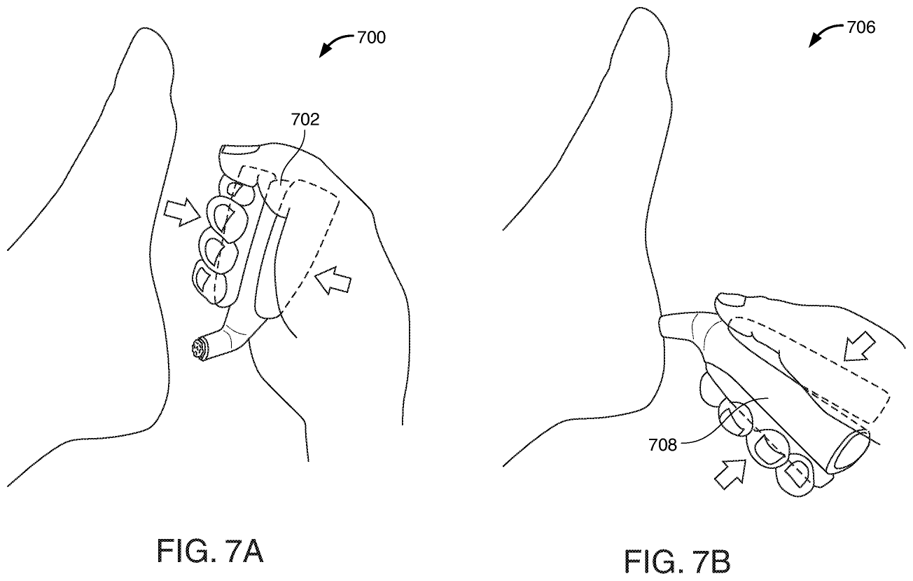

FIG. 8 shows illustrative diagram 800. Illustrative diagram 800 includes plantar wart removal device 100, plantar wart 102, user 106 and a stick-on patch 802. Illustrative diagram 800 may show an initiation of a removal of plantar wart 102.

When user 106 uses plantar wart removal device 100 to initiate removal of plantar wart 102, user 106 may use stick-on patch 802 to protect skin adjacent to plantar wart 102 from the wart removal chemical.

Stick-on patch 802 may be a removable patch. Stick-on patch 802 may include a material. The material may be a mini-closed cell foam and/or any other suitable material. Stick-on patch 802 may include an adhesive backing. The adhesive backing may be a high tack polyurethane adhesive backing. The adhesive backing may be any other suitable adhesive backing.

Stick-on patch 802 may include a die-punched aperture. The die-punched aperture may be placed over plantar wart 102. Stick-on patch 802 may enable plantar wart removal device 100, and/or any other suitable device, to maintain position during a treatment.

Preferably, plantar wart removal device 100 freezes, thaws and refreezes plantar wart 102. When plantar wart 102 is thawed, plantar wart removal device 100 may be removed from plantar wart 102. Stick-on patch 802 may enable plantar wart removal device 100 to be replaced in the same position.

Stick-on patch 802 may include a pull tab. The pull tab may not include an adhesive back. The pull tab may be used to remove stick-on patch 802 from a patient's foot.

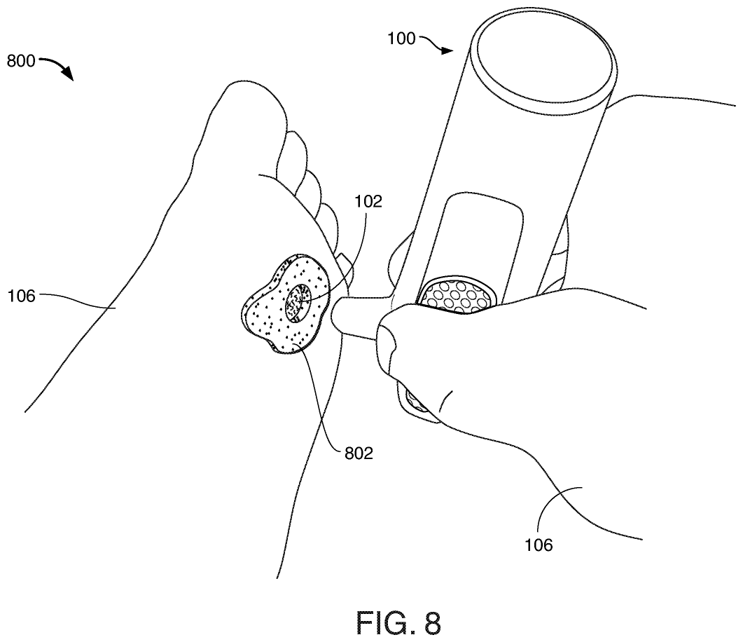

FIG. 9 shows illustrative diagram 900. Illustrative diagram 900 includes plantar wart 102, user 106, plantar wart removal device 902, bendable tip extender 904, circumferential stabilizing support pad 906 and canister 908.

Plantar wart removal device 902 may include bendable tip extender 904. Bendable tip extender 904 may include a material. The material may be thermoplastic elastomer and/or any other suitable material. Bendable tip extender 904 may include a corrugated configuration. Bendable tip extender 904 may be bendable for placement of plantar wart removal device 902 onto plantar wart 102. As such, plantar wart removal device 902 may bend to ensure comfort while initiating removal of plantar wart 102 as well as precise positing of plantar wart removal device 902 on plantar wart 102.

Plantar wart removal device 902 includes circumferential stabilizing support pad 906. Circumferential stabilizing support pad 906 may include a material. The material may be a flexible elastomer and/or any other suitable material. Circumferential stabilizing support pad 906 may be water clear. As such, user 106 may ensure that plantar wart removal device 902 is properly placed on plantar wart 102. Circumferential stabilizing support pad 906 may assist in maintaining perpendicular positioning of plantar wart removal device 902.

It should be noted that circumferential stabilizing support pad 906 may click on, or otherwise engage with, plantar wart removal device 902. Circumferential stabilizing support pad 906 may be removable from plantar wart removal device 902.

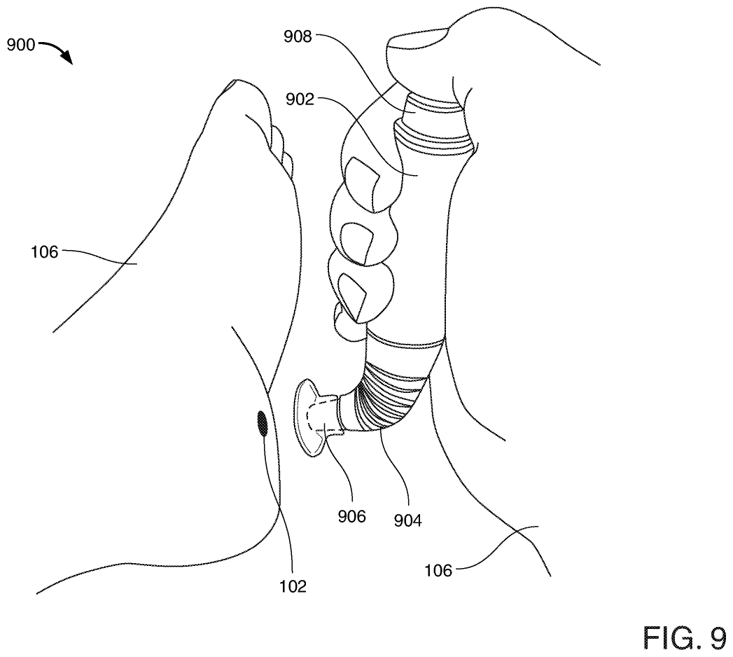

FIG. 10 shows an exemplary application 1000 of cryogenic material using a plantar wart removal device 1002 according to the disclosure.

A collar 1004 preferably includes an applied chrome (or other reflective) finish. Collar 1004 preferably enables sighting of a plantar wart 1008 in alignment with reflected device tip 1005. In obese patients, collar 1004 can be used to overcome excess-weight/bulk-associated difficulties associated with citing the wart and position device tip 1005 vis-vis plantar wart 1008.

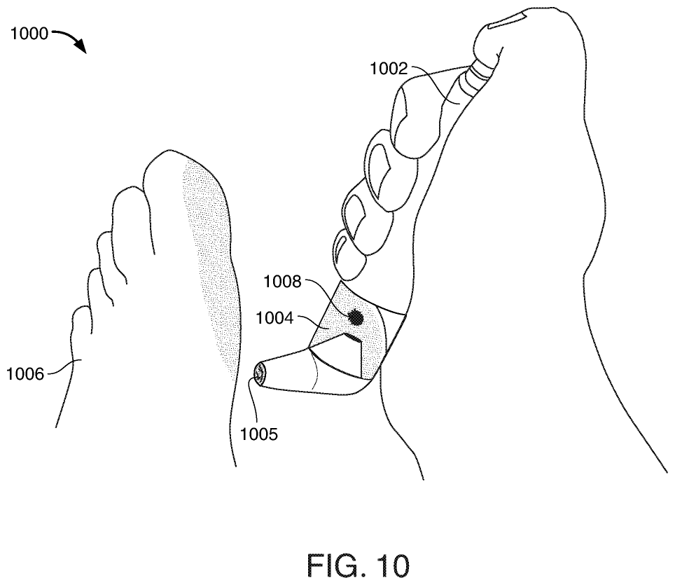

FIG. 11 shows an exemplary application 1000 of cryogenic material using a plantar wart removal device 1002 according to the disclosure.

A collar 1004 preferably includes an applied chrome parabolic mirror surface. The parabolic surface of collar 1004 preferably enables sighting of a plantar wart 1008 in alignment with reflected device tip 1005. In obese patients, collar 1004 can be used to overcome excess-weight/bulk-associated difficulties associated with sighting the wart and position device tip 1005 vis-vis plantar wart 1008/1010.

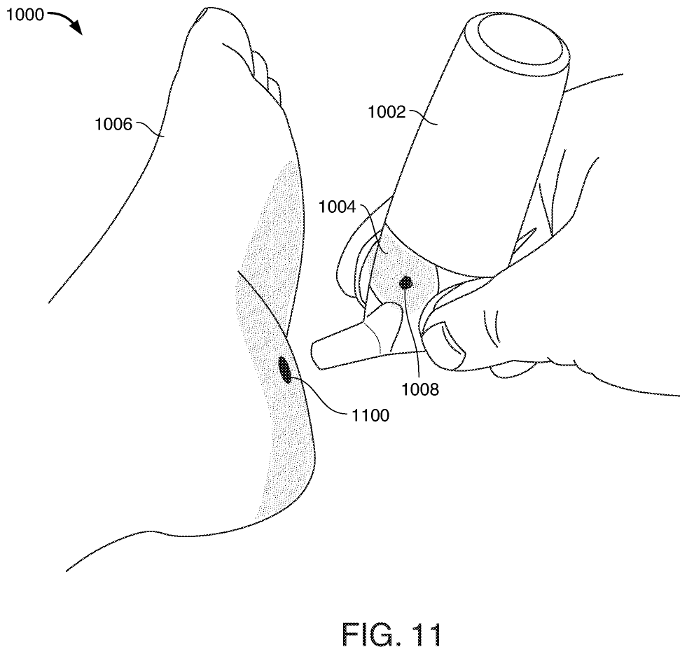

FIG. 12 shows yet another exemplary application 1200 of cryogenic material using a plantar wart removal device 1202 according to the disclosure. Plantar wart removal device 1202 preferably includes a thumb actuated button 1204. In addition, plantar wart removal device 1202 is configured for engagement to cryo-delivery module 1206. Cryo-delivery module 1206 is first mounted on to plantar wart removal device 1202. Then it is prefilled on plantar wart removal device 1202 with cryogenic material. In some embodiments, cryo-delivery module 1206 may be infused with thermochromic ink such that, when cryo-delivery module 1206 undergoes a significant temperature change-e.g., a drop in temperature pursuant to being charged with a cryogenic material, the module may change color from, for example, white to a light blue color.

Thereafter, the pre-filled cryo-delivery module 1206 is affixed onto preferably wafer 1208. Wafer 1208 may be a flexible wafer. Such an engagement is preferably performed in a snapping fashion but other suitable engagements are also possible.

Once cryo-delivery module 1206 is snapped onto wafer 1208, the cryogenic material is unloaded onto the plantar wart 1210 and cryo-delivery module 1206 is removed.

Wafer 1208 may preferably be constructed as a molded plastic cartridge engagement receptacle. Wafer 1208 may be formed from a non-woven poly material. Wafer 1208 may be formed with a high-tack sticky backing such as, for example, a backing formed from a polyurethane adhesive.

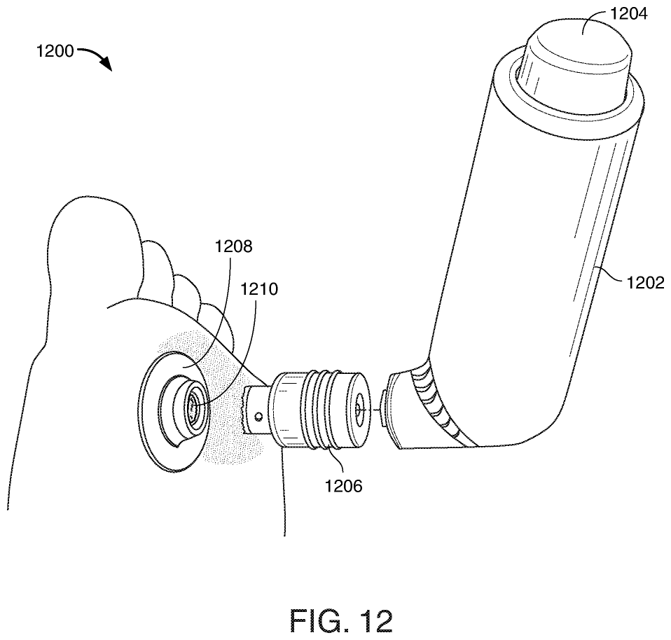

FIGS. 13A-13D show several different applications 1300 of a cryo-delivery module 1306 to a wafer 1310. At 1308, a force is applied to the butt end of a device 1302 for preferably snap-engaging cryo-delivery module 1306 to wafer 1310.

At 13A—device 1302 is shown as prepared for engaging module 1306 to wafer 1310. Button 1304 may be used to release module 1306 and engage it to wafer 1310.

At 13B—device 1302 is shown as engaged to module 1306.

At 13C—module 1306 is mated to wafer 1310 and discharges its cryo-payload onto the plantar wart. Following a full, and possibly multiple, discharges, module 1306 is released from wafer 1310.

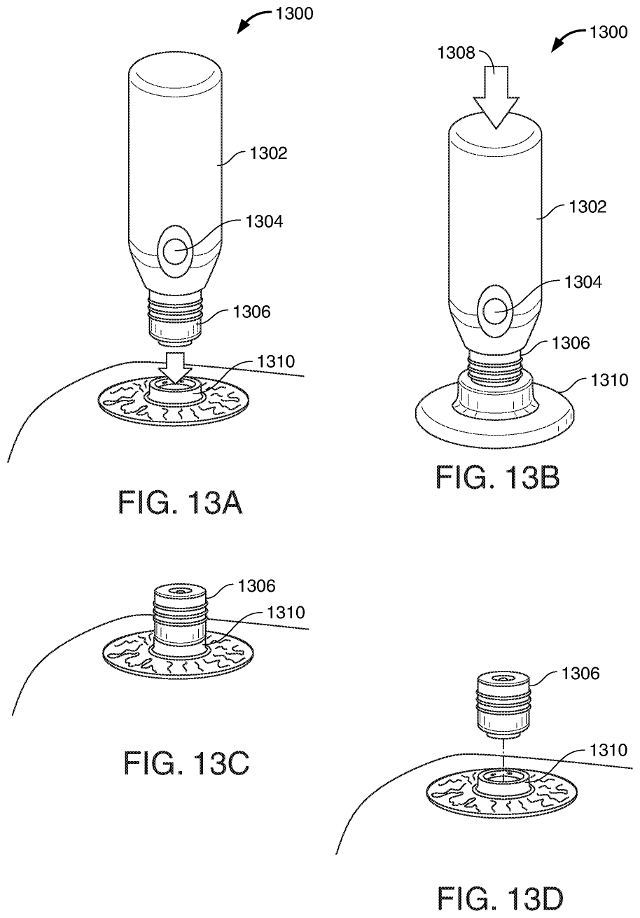

FIG. 14 shows an illustrative diagram of a canister 1406 for insertion into housing 1402. FIG. 14 also shows tip extender 1404 which may include a conduit for delivery of cryogenic material therethrough.

Delivery of the cryogenic material may be transmitted to tip 1408 for application to a plantar wart.

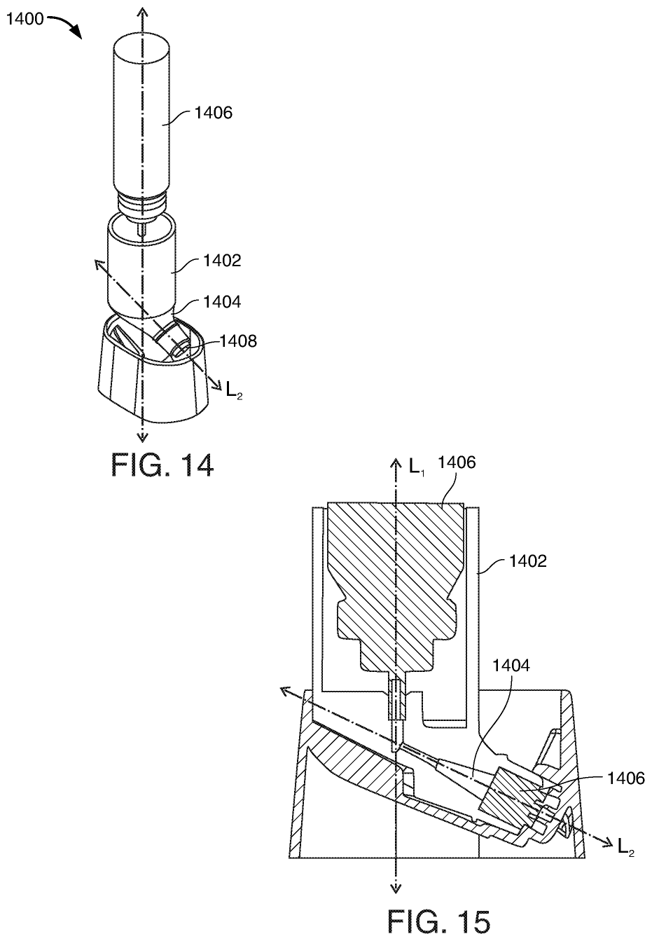

FIG. 15 shows a cross-sectional view of the embodiment shown in FIG. 14. At 1406, a canister is shown. A housing 1402 for receiving canister 1406 is shown. At 1404 a tube for connecting a nozzle output from canister 1406 to a tip 1408 is shown.

Thus, methods and systems for a PLANTAR WART REMOVAL DEVICE may be provided. Persons skilled in the art will appreciate that the present invention can be practiced by other than the described embodiments, which are presented for purposes of illustration rather than of limitation.

What is claimed is:

1. A plantar wart removal device, said plantar wart removal device having a longitudinal axis, said plantar wart removal device comprising:
a canister for holding and selectively dispensing a wart removal chemical on a plantar wart;
a housing, said housing configured to house the canister;
an actuator being mounted on the housing, said actuator configured to cause the canister to dispense the wart removal chemical when actuated;
a tip configured to:
receive the wart removal chemical;
contact the plantar wart; and
deposit the wart removal chemical onto the plantar wart; and
a tip extender:
configured to join the housing with the tip and transmit the wart removal chemical therethrough;
being located at a distal portion of the housing; and
said tip extender having a tip extender longitudinal axis, said tip extender longitudinal axis being between a 30- and 90-degree angle with the longitudinal axis.

2. The plantar wart removal device of claim 1 wherein said plantar wart removal device is configured to enable precise placement and delivery of the wart removal chemical onto the plantar wart.

3. The plantar wart removal device of claim 1 wherein said wart removal chemical comprises cryogenic matter.

4. The plantar wart removal device of claim 1 wherein the tip comprises a thermally conductive material.

5. The plantar wart removal device of claim 1 wherein the housing further comprises a collar, said collar comprising a chrome finish, said collar being located directly above the tip extender.

6. The plantar wart removal device of claim 1 wherein the tip extender is bendable for placement of the plantar wart removal device the plantar wart.

7. A plantar wart removal device, said plantar wart removal device having a longitudinal axis, the plantar wart removal device comprising:
a canister for holding and selectively dispensing a wart removal chemical on a plantar wart;
a housing, said housing configured to house the canister;
an actuator being mounted on the housing, said actuator configured to cause the canister to dispense the wart removal chemical when actuated;
a tip nozzle, mounted on the canister, for depositing the wart removal chemical;
a tip enclosure configured to focus output of the tip nozzle, the tip enclosure:
located at a distal portion of the housing; and
having a tip enclosure longitudinal axis, said tip enclosure longitudinal axis being between a 30- and 90-degree angle with the longitudinal axis.

8. The plantar wart removal device of claim 7 wherein said plantar wart removal device is configured to remove plantar warts unaided.

9. The plantar wart removal device of claim 7 wherein said wart removal chemical comprises cryogenic matter.

10. The plantar wart removal device of claim 7 wherein the tip enclosure comprises a polymeric material.

11. The plantar wart removal device of claim 7 wherein the tip nozzle is a spray nozzle.

12. The plantar wart removal device of claim 7 wherein the output is the wart removal chemical.

13. The plantar wart removal device of claim 7 wherein the housing further comprises a collar, said collar comprising a chrome finish, said collar being located directly above the tip enclosure.

14. A plantar wart removal device, said plantar wart removal device having a longitudinal axis, said plantar wart removal device for initiating removal of a plantar wart, the plantar wart removal device comprising:
a canister for holding and selectively dispensing a cryogenic material;
a housing, said housing configured to house the canister;
a receptacle, said receptable configured to cause the canister to selectively dispense the cryogenic material when engaged with the plantar wart removal device; and
an absorbent tip configured to:
absorb and store the cryogenic material from the canister; and
deposit the cryogenic material onto the plantar wart; and
a tip extender:
configured to join the housing with the absorbent tip and transmit the cryogenic material therethrough;
being located at a distal portion of the housing; and
said tip extender having a tip extender longitudinal axis, said tip extender longitudinal axis being between a 30- and 90-degree angle with the longitudinal axis.

15. The plantar wart removal device of claim 14 wherein said plantar wart removal device is configured to remove plantar warts unaided.

16. The plantar wart removal device of claim 14 wherein the plantar wart removal device is configured to automatically deliver, when engaged with the receptacle, multiple applications of the cryogenic material.

17. The plantar wart removal device of claim 14 wherein the plantar wart removal device is configured to release, for a predetermined amount of time, the cryogenic material upon engagement with the receptacle.

18. The plantar wart removal device of claim 14 wherein the plantar wart removal device is configured to automatically:
deposit the cryogenic material onto the plantar wart, thereby freezing the plantar wart;
thaw the plantar wart; and
refreeze the plantar wart by depositing the cryogenic material a second time.

19. The plantar wart removal device of claim 18 wherein the plantar wart removal device is configured to automatically:
freeze the plantar wart for a duration of between 15 and 25 seconds;
thaw the plantar wart for a duration of between 20 and 40 seconds; and
refreeze the plantar wart for a duration of between 15 and 25 seconds.

20. A plantar wart removal method, said plantar wart removal method using a plantar wart removal device, said plantar wart removal device having a longitudinal axis, said plantar wart removal method comprising:
holding and selectively dispensing a cryogenic material, said cryogenic material being stored, pre-application, in a canister, said canister being located in a housing;
causing the canister, using an actuator mounted on the housing, to selectively dispense the cryogenic material when actuated;
absorbing the cryogenic material in an absorbent tip and storing the cryogenic material in the absorbent tip;
applying, for between 15 and 25 seconds, the absorbent tip to a plantar wart to freeze the plantar wart;
allowing the plantar wart to thaw for a duration of between 20 and 40 seconds; and
reapplying, for a duration of between 15 and 25 seconds, the absorbent tip to refreeze the plantar wart.
